(19) United States
(12) Patent Application Publication
(10) Pub. No.: US 2017/0252093 A1
(43) Pub. Date: Sep. 7, 2017

(54) DEVICE AND METHOD FOR ENERGIZING SURGICAL INSTRUMENTS

(71) Applicant: COVIDIEN LP, Mansfield, MA (US)

(72) Inventors: DUANE E. KERR, LOVELAND, CO (US); JAMES D. ALLEN, IV, BROOMFIELD, CO (US)

(21) Appl. No.: 15/444,651

(22) Filed: Feb. 28, 2017

Related U.S. Application Data
(60) Provisional application No. 62/301,839, filed on Mar. 1, 2016.

(51) Int. Cl. A61B 18/14

(52) U.S. Cl. CPC A61B 18/1442; A61B 2018/1405; A61B 2018/146; A61B 2018/1452; A61B 2218/00

(57) ABSTRACT

A surgical instrument includes a housing having an end effector operably coupled thereto and configured to mechanically engage tissue. The instrument further includes an energy transfer switch disposed on the housing and configured to operably engage a tip of an electrosurgical instrument such that upon activation of the electrosurgical instrument, electrosurgical energy is transmitted to the end effector of the surgical instrument to treat tissue in an electrosurgical fashion.

### CROSS REFERENCE TO RELATED APPLICATION

The present application claims the benefit of and priority to U.S. Provisional Application Ser. No. 62/301,839, filed on Mar. 1, 2016, the entire contents of which are incorporated herein by reference.

### BACKGROUND

### Technical Field

The present disclosure relates to surgical instruments and, more particularly, to surgical devices and methods for energizing surgical instruments.

### Background of Related Art

Electrosurgical instruments are widely used in surgical procedures. The majority of these instruments are connected in some fashion to an electrosurgical generator via a cord or cable. Generally, electrosurgical instruments may be classified into two categories, namely bipolar instruments and monopolar instruments.

Bipolar surgical instruments typically include two generally opposing electrodes charged to different electric potentials to selectively apply energy to tissue. Bipolar electrosurgical forceps, for example, utilize both mechanical clamping action and electrical energy to effect hemostasis by heating tissue to coagulate and/or cauterize tissue. Monopolar surgical instruments, on the other hand, include an active electrode, and are used in conjunction with a remote return electrode, e.g., a return pad, to apply energy to tissue. Monopolar instruments have the ability to rapidly move through tissue and dissect through narrow tissue planes.

In some surgical procedures, it may be beneficial to use both bipolar and monopolar instrumentation, e.g., procedures where it is necessary to dissect through one or more layers of tissue in order to reach underlying tissue(s) to be treated. Further, it may be beneficial, particularly with respect to endoscopic surgical procedures, to provide a single instrument incorporating both bipolar and monopolar features, thereby obviating the need to alternatingly remove and insert the bipolar and monopolar instruments in favor of one another. However, a dual bipolar and monopolar instrument may prove impractical in some circumstances since it is not always desirable to utilize the same type of instrument for a given surgical procedure, e.g., an electrosurgical pencil may be desirable in one instance (cutting, blending or spot coagulation) and an electrosurgical forceps in another (sealing). Moreover, introducing a second (or third) electrosurgical instrument into the operating field may in some circumstance prove impractical (too many cords, may require a second generator, expensive, etc.).

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

A surgical instrument provided in accordance with aspects of the present disclosure includes a housing having an end effector operably coupled to the housing and configured to mechanically engage tissue. The instrument further includes an energy transfer switch configured to operably engage a tip of an electrosurgical instrument such that upon activation of the electrosurgical instrument, electrosurgical energy is transmitted to the end effector of the surgical instrument to treat tissue in an electrosurgical fashion. The energy transfer switch may be disposed on any part of the instrument including the housing, the handle, the shaft, the end effector, etc.

In aspects, the energy transfer switch includes a recess defined therein that supports an electrical contact configured to engage the tip of the electrosurgical instrument. The electrical contact may be coupled to a conduit that transmits electrosurgical energy to the end effector. In aspects, the electrical contact is ring-shaped.

In other aspects, the energy transfer switch includes a mechanical interface that is configured to mate with a corresponding mechanical interface disposed on the electrosurgical instrument to facilitate electromechanical connection therebetween. In one embodiment, the energy transfer switch includes a universal connector to facilitate electromechanical engagement with tips of varying electrosurgical instruments.

In other aspects, the surgical instrument includes a shaft extending from a distal end of the housing that operably engages the end effector and the energy transfer switch is disposed on the shaft.

In aspects, the energy transfer switch transmits energy of a first potential to the end effector so the user can selectively treat tissue in a monopolar fashion.

A surgical instrument provided in accordance with other aspects of the present disclosure includes a housing having an end effector operably coupled to the housing and configured to mechanically engage tissue. The end effector includes first and second treatment members. An energy transfer switch is included and is configured to operably engage a tip of an electrosurgical instrument such that upon activation of the electrosurgical instrument, electrosurgical energy is transmitted to the end effector of the surgical instrument to treat tissue in an electrosurgical fashion. The energy transfer switch includes a first connector coupled to a first conduit for transmitting energy of a first potential to the first treatment member of the end effector and a second connector coupled to a second conduit for transmitting energy of a second potential to the second treatment member of the end effector.

In aspects, the energy transfer switch includes a recess defined therein that supports a pair of electrical contacts configured to engage the tip of the electrosurgical instrument. The pair of electrical contacts may form a ring separated by an electrical insulator.

In aspects, the energy transfer switch includes a mechanical interface that is configured to mate with a corresponding mechanical interface disposed on the electrosurgical instrument to facilitate electromechanical connection therebetween. The energy transfer switch may include a universal connector to facilitate electromechanical engagement with tips of varying electrosurgical instruments.

The present disclosure also relates to a method of energizing a surgical instrument and includes introducing an electrosurgical instrument into an operating field or utilizing an electrosurgical instrument in an operating field. The method also includes: engaging an electrified component of the electrosurgical instrument to an energy transfer switch of an additional surgical instrument; energizing the electrosurgical instrument to provide energy to the energy transfer switch and to a treatment member of the additional surgical instrument; treating tissue with the additional surgical instrument; and disengaging the electrified component from the energy transfer switch of the additional surgical instrument.

### BRIEF DESCRIPTION OF THE DRAWINGS

Various aspects and features of the present disclosure described herein with reference to the drawings wherein:

FIG. 1 is a perspective view of an electrosurgical system according to the present disclosure including a generator and electrosurgical pencil;

FIGS. 2A-2C are perspective views of various electrosurgical instruments for use with the electrosurgical system of FIG. 1 each including an electrical connection disposed thereon;

FIG. 3A is an enlarged, schematic cross section of the electrical connection of FIGS. 2A-2C with a monopolar functionality;

FIG. 3B is an enlarged, schematic cross section of an alternate electrical connection of FIGS. 2A-2C having a bipolar functionality;

FIG. 4 is a flow chart detailing a method according to the present disclosure; and

FIG. 5 is a schematic illustration of a robotic surgical system configured for use in conjunction with aspects and features of the present disclosure.

### DETAILED DESCRIPTION

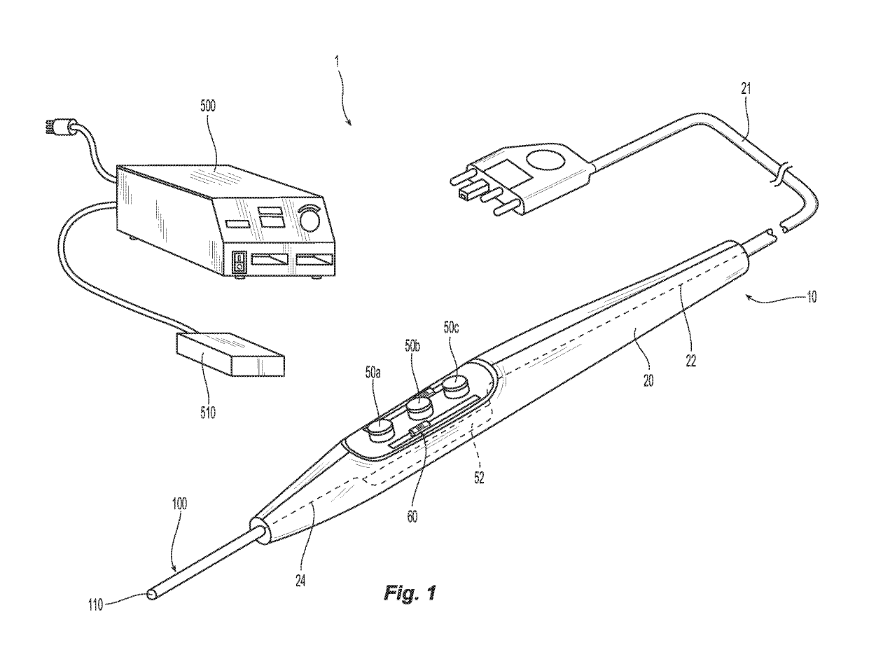

FIG. 1 sets forth a perspective view of an electrosurgical system 1 that includes an electrosurgical pencil 10 and a generator 500. While the following description will be directed towards electrosurgical pencils, the features and concepts (or portions thereof) of the present disclosure can be applied to any electrosurgical type instrument, e.g., forceps, suction coagulator, vessel sealers, etc. as long as an appropriate tip or electromechanical mechanical interface is configured to mate with a corresponding energy transfer switch of a second or additional instruments.

Electrosurgical pencil 10 includes an elongated housing 20 configured and adapted to support an end effector 100 at a distal end thereof which may be in the form of a cylindrical tip, blade, loop, needle or ball 110. A distal portion of the tip 110 extends from the housing 20 while a proximal portion is selectively retained within the housing 20.

As shown, electrosurgical pencil 10 is coupled to a conventional electrosurgical generator 500 via a cable 21 which ultimately electrically interconnects electrosurgical generator 500 with the tip 110. Cable 21 may further include one or more control wires 22 which electrically interconnect one or more mode activation switches, e.g., switches 50a-50c (described in greater detail below), supported on housing 20, with electrosurgical generator 500. For the purposes herein the terms “switch” or “switches” includes electrical actuators, mechanical actuators, electro-mechanical actuators (rotatable actuators, pivotable actuators, toggle-like actuators, buttons, etc.) or optical actuators.

Electrosurgical activation switches 50a-50c are each supported on an outer surface of housing 20. Each activation switch 50a-50c is operatively connected to a location on a circuit 52 (shown in phantom in FIG. 1) which, in turn, controls the transmission of RF electrical energy supplied from generator 500 to electrosurgical tip 110. In embodiments, the circuit 52 maybe operatively connected to a “voltage divider network” or “VDN” which forms a switch closure. For the purposes herein, the term “voltage divider network” relates to any known form of resistive, capacitive or inductive switch closure (or the like) which determines the output voltage across a voltage source (e.g., one of two impedances) connected in series. A “voltage divider” as used herein relates to a number of resistors connected in series which are provided with taps at certain points to make available a fixed or variable fraction of the applied voltage.

In use, depending on which activation switch 50a-50c is depressed a respective switch is pressed into contact with the VDN of the circuit 52 and a characteristic signal is transmitted to electrosurgical generator 500 via one or more control wires 22. Each activation switch 50a-50c may be configured and adapted to control the mode and/or “waveform duty cycle” to achieve a desired surgical intent. For example, activation switch 50a can be set to deliver a characteristic signal to electrosurgical generator 500 which in turn transmits a duty cycle and/or waveform shape which produces a cutting and/or dissecting effect/function. Meanwhile, activation switch 50b can be set to deliver a characteristic signal to electrosurgical generator 500 which in turn transmits a duty cycle and/or waveform shape which produces a blending effect/function (e.g., a combination of a dissecting and a hemostatic effect/function). Finally, activation switch 50c can be set to deliver a characteristic signal to electrosurgical generator 500 which in turn transmits a duty cycle and/or waveform shape which produces a hemostatic effect/function.

Electrosurgical pencil 10 further includes an intensity controller 60 slidingly supported on housing 20. Intensity controller 60 includes a pair of sliders supported on opposite sides of the switches 50a-50c which are easily manipulatable by right or left handed users. Intensity controller 60 is a slide potentiometer wherein a proximal-most position corresponds to a relative low intensity setting and a distal-most position corresponds to a relative high intensity setting. A plurality of intermediate positions between the proximal-most and distal-most positions corresponds to various intermediate intensity settings. Various positions of the intensity controller 60 may include tactile feedback (e.g., a series of cooperating detents) that provide a series of discreet energy intensities from a low intensity setting to a high intensity setting. The series of cooperating discreet or detented positions provides the surgeon with a degree of tactile feedback relating to the energy control.

Intensity controller 60 may be configured and adapted to adjust the power parameters (e.g., voltage, power and/or current intensity) and/or the power verses impedance curve shape to affect the perceived output intensity. For example, the greater intensity controller 60 is displaced in a distal direction the greater the level of the power parameters transmitted to electrosurgical tip 110.

During conventional use and depending on the particular electrosurgical function desired, the surgeon depresses one of activation switches 50a-50c to thereby transmitting a respective characteristic signal to electrosurgical generator 500. In turn, electrosurgical generator 500 transmits an appropriate waveform output to electrosurgical tip 110 via a transmission wire 24. In order to vary the intensity of the power parameters of electrosurgical pencil 10, the surgeon moves the intensity controller 60 as needed to increase the intensity of the waveform.

As described above, intensity controller 60 can be configured and adapted to provide a degree of tactile feedback. Alternatively, audible feedback can be produced from intensity controller 60 (e.g., a “click”), from electrosurgical energy source 500 (e.g., a “tone”) and/or from an auxiliary sound-producing device such as a buzzer (not shown).

As mentioned above, if a second (or additional instruments) is needed during a particular surgical procedure, the new instrument is typically connected to the generator 500 resulting in the electrosurgical pencil 10 being unplugged. The generator 500 may then have to be recalibrated to accommodate the second or additional instrument. Alternatively, some electrosurgical generators 500 may allow multiple instruments to be connected at the same time eliminating the need to unplug and recalibrate, however, a second cord is still typically required to connect the second instrument.

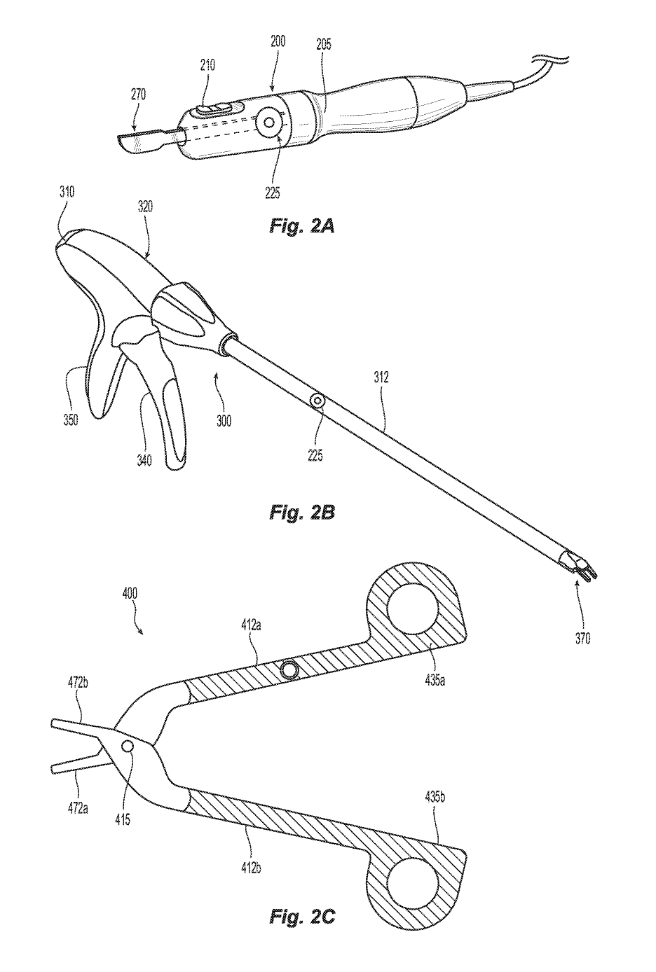

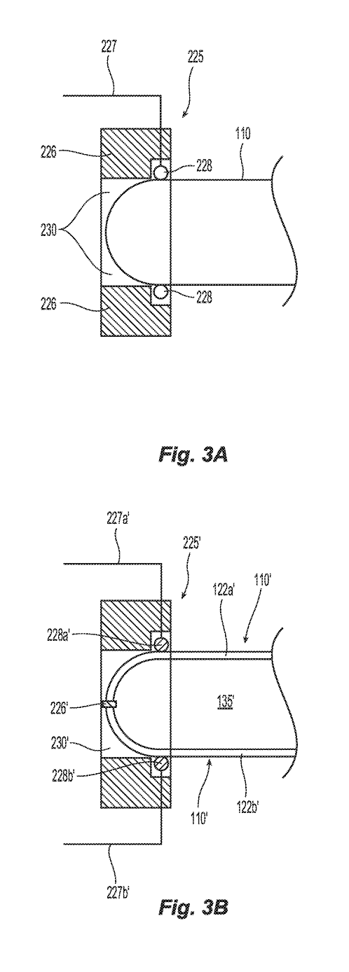

FIGS. 2A-2C show various embodiments of second or additional instruments according to one aspect of the present disclosure for use with pencil 10. More particularly, FIG. 2A shows an electrosurgical knife 200 including a housing 205 that supports an electrosurgical blade 270 at a distal end thereof. An activation switch 210 is also supported on the housing 205 which allows a surgeon to selectively energize the blade 270 as needed. An energy transfer switch 225 is included on housing 205 and is configured to electromechanically engage the tip 110 of the electrical pencil 10 to energize the knife 200 for electrical use. Energy transfer switch 225 may be disposed on other suitable parts of the knife 200 to accomplish the same or similar purpose. Details of the energy transfer switch 225 are described below with reference to FIGS. 3A and 3B.

FIG. 2B shows an endoscopic surgical forceps 300 including a housing 320 having an integral handle 350 that extends therefrom and a movable handle 340 that is selectively actuatable relative to handle 350 to operate end effector 370. The housing 320 includes a shaft 312 that extends from a distal end thereof that supports energy transfer switch 225. Energy transfer switch 225 may be disposed on other suitable parts of the forceps 300 to accomplish the same or similar purpose. In this instance, energy transfer switch 225 is disposed on shaft 312 and is configured to electromechanically engage the tip 110 of the electrical pencil 10 to energize the forceps 300 for electrical use.

FIG. 2C shows an open surgical forceps 400 that includes a pair of opposing shaft members 412a and 412b each having a handle 435a and 435b at a proximal end thereof and a jaw member 472a and 472b at a distal end thereof, respectively. Handles 435a and 435b are selectively moveable relative to one another about a pivot 415 to move the jaw members 472a and 472b from an open, approximated position for manipulating tissue and a closed, grasping position for treating tissue therebetween. In this instance, energy transfer switch 225 is included on one or both shafts, e.g., shaft 412a, and is configured to electromechanically engage the tip 110 of the electrical pencil 10 to energize the forceps 400 for electrical use. Energy transfer switch 225 may be disposed on other suitable parts of the forceps 400 to accomplish the same or similar purpose.

FIGS. 3A and 3B show two embodiments of the energy transfer switch 225 for use with the instruments shown in FIGS. 2A-2C. The energy transfer switch 225 may be disposed on any part of the additional instrument for energy transfer. Aspects of the energy transfer switch 225 described herein and the use thereof may apply to other types of surgical instruments not necessarily show or described herein. As such, the various instruments shown in FIGS. 2A-2C are shown merely as examples and should not be construed as limiting.

FIG. 3A shows one embodiment of the energy transfer switch 225 that may be used to transfer monopolar energy or energy having one potential to a surgical instrument, e.g., knife 200, shown in FIG. 2A. More particularly, energy transfer switch 225 is housed within a recess 230 defined within the surgical instrument, e.g., housing 205 of knife 200. Energy transfer switch 225 includes an insulative outer washer 226 that secures a conductive inner ring contact 228 therein. Inner ring contact 228 may be dimensioned to include a diameter suitable to mechanically and electrically engage the tip 110 of pencil 10 or may be configured to accept varying-sized tips 110 depending upon a particular purpose. Any type of electromechanical connector or contact known in the art may be utilized for this purpose. Energy transfer switch 225 also includes an electrical conduit 227 that is configured to transfer energy to the blade 270. Conduit 227 may be a wire or any other type of conduit, e.g., tube or sleeve, configured to carry electrical energy to the blade 270 upon activation of switch 210.

In use, the surgeon while utilizing a surgical instrument with an electrosurgical monopolar tip, e.g., pencil 10 with tip 110, may at any time during the surgical procedure introduce one or more additional instruments into the operating field for use. In embodiments, a typical surgical instrument may be introduced and electrified to electrosurgically treat tissue, an electrosurgical instrument may be introduced to electrosurgically treat tissue, or a combination electrosurgical and mechanical instrument may be introduced, e.g., electrosurgical forceps with mechanical cutter. To activate the additional instrument, the surgeon engages the tip, e.g., tip 110 of pencil 10, within recess 230 defined within the energy transfer switch 225 and activates the pencil 10. The treatment portion may be immediately energized for treatment of tissue. Alternatively, as in the case with knife 200, the switch 210 of the knife 200 may then be activated to utilize the blade 270 to electrically treat tissue.

In embodiments, switch 210 may be turned to an open position such that energy is transferred to the blade 270 upon activation of the pencil 10. In other embodiment, the knife 200 may not include a switch 210 and activation of the blade 270 would be controlled by the controls on the pencil 10. In embodiments, the switches 50a-50c controlling the various waveforms mentioned above and the intensity controller 60 of the pencil 10 may be transferred to the electrical output of the blade 270.

As can be appreciated, the tip 110 of pencil 10 (or some other surgical instrument as mentioned above) may be configured to include a first mechanical interface and the inner contact 228 of the energy transfer switch 225 may be configured to include a second mechanical interface to facilitate electromechanical connection therebetween. As such, a line of instrumentation may be manufactured to include an energy transfer switch 225 that works with the tip 110 of the pencil 110. In other embodiments, the additional instruments may be more versatile. For example, the energy transfer switch 225 may include a variable or universal mechanical interface, e.g., inner connector 228, so as to allow engagement of a variety of different tip, knife or jaw geometries and diameters.

In embodiments, the energy transfer switch 225 may include one or more safety mechanisms (not shown) to prevent accidental activation of the blade 270 upon engagement of the tip 110 with the inner contact 228. For example, switch 210 may default to a closed position upon engagement of the tip 110 with the inner contact 228. Various known mechanical, electrical or electromechanical configurations may be suitable to accomplish this purpose. Other safety features may include an automated shut-off should the mechanical or electrical connection between the tip 110 and the inner contact 228 get compromised during use. Various known safety features may be employed to prevent or at least warn of this issue, e.g., locking mechanisms, sensors (force or strain gauges on inner contact 228), audible or tactile cues, etc.

FIG. 3B shows an alternate embodiment of an energy transfer switch 225′ for use with tip 110′ of bipolar electrosurgical pencil 10. Tip 110 is similar to tip 110 and for the purposes of brevity only those features that are different are described herein. Tip 110′ includes an inner insulator 135′ surrounded by two opposing electrical conductors 122a′ and 122b′. Conductors 122a′ and 122b′ may be embedded or etched within the insulator 135′, disposed atop the insulator 135′ or engaged in some other fashion. Conductors 122a′ and 122b′ are configured to carry first and second electrical potentials, respectively, for the bipolar treatment of tissue when utilizing the pencil 10. A second insulator 126′ is positioned between the conductors 122a′ and 122b′ and electrically isolates the two conductors 122a′ and 122b′ during bipolar treatment of tissue.

Energy transfer switch 225′ is similar to the energy transfer switch 225 shown in FIG. 3A and only those elements that are different are described herein. Energy transfer switch 225′ includes first and second energy conduits 227a′ and 227b′ positioned on opposing sides of the switch 225′ and configured to engage respective contacts 122a′ and 122b′. More particularly, as tip 110′ is inserted into recess 230′ of switch 225′, contacts 122a′ and 122b′ align with corresponding contacts 228a′ and 228b′ and, upon activation, respective first and second electrical potentials are transferred to conduits 227a′ and 227b′ to provide energy to the treatment members, of the additional surgical instrument, e.g., jaw members 472a and 472b of forceps 400. Using FIG. 2C as an example, the first potential may be transferred to a first treatment member, e.g., jaw member 472a and the second electrical potential may be transferred to a second treatment member, e.g., jaw member 472b thereby allowing the forceps 400 to treat tissue in a bipolar manner. Various types of bipolar instruments may be utilized in this fashion wherein energy of a first potential is transmitted to the first treatment member (or first pole of the end effector) and energy of a second potential is transmitted to the second treatment member (or second pole of the end effector).

In use, the surgeon while utilizing the electrosurgical bipolar tip 110′ of the pencil 10, may at any time during the surgical procedure introduce one or more additional instruments into the operating field for use. To activate the additional instrument(s), the surgeon engages the tip 110′ within recess 230′ and activates the pencil 10. This may then energize the additional instrument to treat tissue in a bipolar manner, e.g., provide first and second electrical potential to the jaw members 472a and 472b of forceps 400, or may require the surgeon to activate a second switch (disposed on the additional instrument, e.g., switch 210 of knife 200) to treat tissue. Alternatively, once the energy transfer switch 225′ is engaged, an electrical or electromechanical protocol may be established that transfers electrical control to the pencil 10 for activating the additional instrument, e.g., knife 200. Similar to the embodiments described above, energy transfer switch 225′ may include one or more of the above-mentioned safety mechanisms to prevent accidental activation upon engagement of the tip 110′ with the inner contacts 228a′ and 228b′. For example, switch 210 may default to a closed position upon engagement of the tip 110′ with the inner contacts 228a and 228b. Other safety features may include an automated shut-off should the mechanical or electrical connection between the tip 110′ and the inner contacts 228 and/or 228b get compromised during use. Various known safety features may be employed to prevent or at least warn of this issue, e.g., locking mechanisms, sensors (force or strain gauges on inner contacts 228a and 228b), audible or tactile cues, etc.

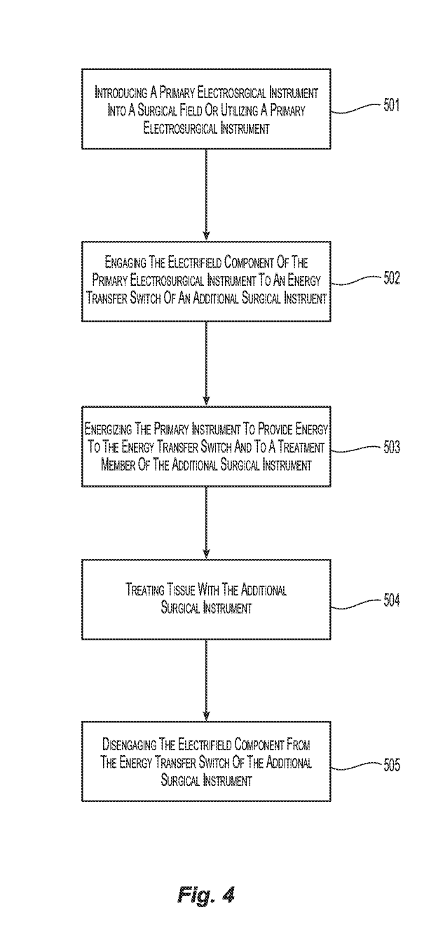

The present disclosure also relates to a method for energizing a secondary surgical instrument for electrical use that is introduced into the operating field and is shown in the flow chart of FIG. 4. The method includes the initial step 501 of introducing a primary electrosurgical instrument, e.g., electrosurgical pencil 10, into a surgical field or utilizing a primary electrosurgical instrument, e.g., pencil 10, during a surgical procedure. As mentioned above, either a bipolar or monopolar instrument may be utilized for this purpose. The method also includes: step 502—engaging the electrified component, e.g., tip 110, 110′, of the primary electrosurgical instrument, e.g., pencil 10, to an energy transfer switch, e.g., switch 225 or 225′, of an additional surgical instrument, e.g., knife 200; step 503—energizing the primary instrument, e.g., pencil 10, to provide energy to the energy transfer switch 225, 225′ and to a treatment member, e.g., blade 270 of the additional surgical instrument; step 504—treating tissue with the additional surgical instrument 200; and step 505—disengaging the electrified component, e.g., tip 110, 110′, from the energy transfer switch 225, 225′ of the additional surgical instrument.

As mentioned above, the additional surgical instrument may include a separate switch, e.g., switch 210 or knife 200, that is disabled upon engagement of the tip 110, 100′ with the energy transfer switch 225, 225′. Alternatively, the switch 210 may act as a safety mechanism and allow the surgeon a greater degree of surgical control depending upon a particular purpose.

The various surgical instruments may also be manufactured as a system or sold as a kit. For example, the electrosurgical pencil 10 may be manufactured for use in surgery and sold along with one or more of the additional instruments shown herein (or perhaps another type of instrument not shown herein). The additional instrument may be used strictly mechanically or electrified to treat tissue in either a monopolar or bipolar fashion as described above. Moreover, the energy transfer switch of the additional surgical instrument may include a universal coupler that is configured to receive any tip of any instrument and convert that energy to the appropriate energy for the additional surgical instrument. The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

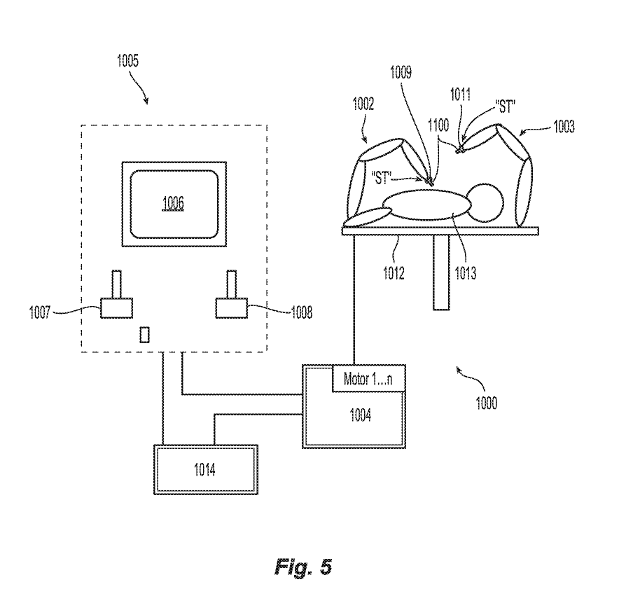

Referring initially to FIG. 5, a medical work station is shown generally as work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus the surgical tool (including end effector 1100) execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of end effector 1100. Medical work station 1000 may also include more than two robot arms 1002, 1003, the additional robot arms likewise being connected to control device 1004 and being telemanipulatable by means of operating console 1005. A medical instrument or surgical tool (including an end effector 1100) may also be attached to the additional robot arm. Medical work station 1000 may include a database 1014, in particular coupled to with control device 1004, in which are stored, for example, pre-operative data from patient/living being 1013 and/or anatomical atlases.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed is:

1. A surgical instrument, comprising:
a housing;
an end effector operably coupled to the housing and configured to mechanically engage tissue; and
an energy transfer switch disposed on the housing and configured to operably engage a tip of an electrosurgical instrument such that upon activation of the electrosurgical instrument, electrosurgical energy is transmitted to the end effector of the surgical instrument to treat tissue in an electrosurgical fashion.

2. The surgical instrument of claim 1 wherein the energy transfer switch includes a recess defined therein that supports an electrical contact configured to engage the tip of the electrosurgical instrument.

3. The surgical instrument of claim 2 wherein the electrical contact is coupled to a conduit that transmits electrosurgical energy to the end effector.

4. The surgical instrument of claim 1 wherein the energy transfer switch includes a mechanical interface that is configured to mate with a corresponding mechanical interface disposed on the electrosurgical instrument to facilitate electromechanical connection therebetween.

5. The surgical instrument of claim 1 wherein the energy transfer switch includes a universal connector to facilitate electromechanical engagement with tips of varying electrosurgical instruments.

6. The surgical instrument of claim 2 wherein the electrical contact is ring-shaped.

7. The surgical instrument of claim 1 wherein the surgical instrument includes a shaft extending from a distal end of the housing and operably engaged to the end effector, and wherein the energy transfer switch is disposed on the shaft.

8. The surgical instrument of claim 1 wherein the energy transfer switch transmits energy of a first potential to the end effector so the user can selectively treat tissue in a monopolar fashion.

9. A surgical instrument, comprising:
a housing;
an end effector operably coupled to the housing and configured to mechanically engage tissue, the end effector including first and second treatment members; and
an energy transfer switch configured to operably engage a tip of an electrosurgical instrument such that upon activation of the electrosurgical instrument, electrosurgical energy is transmitted to the end effector of the surgical instrument to treat tissue in an electrosurgical fashion, the energy transfer switch including:
a first connector coupled to a first conduit for transmitting energy of a first potential to the first treatment member of the end effector; and
a second connector coupled to a second conduit for transmitting energy of a second potential to the second treatment member of the end effector.

10. The surgical instrument of claim 9 wherein the energy transfer switch includes a recess defined therein that supports a pair of electrical contacts configured to engage the tip of the electrosurgical instrument.

11. The surgical instrument of claim 10 wherein the pair of electrical contacts form a ring and are separated by an electrical insulator.

12. The surgical instrument of claim 9 wherein the energy transfer switch includes a mechanical interface that is configured to mate with a corresponding mechanical interface disposed on the electrosurgical instrument to facilitate electromechanical connection therebetween.

13. The surgical instrument of claim 9 wherein the energy transfer switch includes a universal connector to facilitate electromechanical engagement with tips of varying electrosurgical instruments.

14. A method of energizing a surgical instrument, comprising:
introducing an electrosurgical instrument into an operating field;
engaging an electrified component of the electrosurgical instrument to an energy transfer switch of an additional surgical instrument;
energizing the electrosurgical instrument to provide energy to the energy transfer switch and to a treatment member of the additional surgical instrument;
treating tissue with the additional surgical instrument; and
disengaging the electrified component from the energy transfer switch of the additional surgical instrument.
